SEQUENCE LISTING (36 sequences)

SEQ ID NO: 1 - DNA, 21 bp, Artificial Sequence
  Description of Artificial Sequence synthetic primer
gattggcgctcctcgttcacc

SEQ ID NO: 2 - DNA, 50 bp, Artificial Sequence
  Description of Artificial Sequence synthetic primer
ccattatttttctagtcgacatggcatcgattcccaaggccaatgtcccc

SEQ ID NO: 3 - DNA, 25 bp, Artificial Sequence
  Description of Artificial Sequence synthetic primer
cacagagaatgtgccgtttctttgg

SEQ ID NO: 4 - DNA, 24 bp, Artificial Sequence
  Description of Artificial Sequence synthetic primer
tcacatatcccctactcccgagcc

SEQ ID NO: 5 - DNA, 45 bp, Artificial Sequence
  Description of Artificial Sequence synthetic primer
gttacacgctttgattctgtgggtaccgatgttatattcagctac

SEQ ID NO: 6 - DNA, 44 bp, Artificial Sequence
  Description of Artificial Sequence synthetic primer
cccaatagcggccgcagttgataatatcaatatctaaaactccc

SEQ ID NO: 7 - DNA, 42 bp, Artificial Sequence
  Description of Artificial Sequence synthetic primer
ggcatatacgagcatggtaccagggacagatgcccatccttg

SEQ ID NO: 8 - DNA, 40 bp, Artificial Sequence
  Description of Artificial Sequence synthetic primer
gtataaaaggggagggtaccgggaaagatttgtgggcctg

SEQ ID NO: 9 - DNA, 45 bp, Artificial Sequence
  Description of Artificial Sequence synthetic primer
gtatgtagctgcggccgcctccgtcttcacttcttcgcccgcact

SEQ ID NO: 10 - DNA, 50 bp, Artificial Sequence
  Description of Artificial Sequence synthetic primer
ccgccttcctcactaaccggccggcaggtaccgatggactcagcattatc

SEQ ID NO: 11 - DNA, 47 bp, Artificial Sequence
  Description of Artificial Sequence synthetic primer
ctctagaatgctacggccgttcgaggtaccttataggaaaaaggtag

SEQ ID NO: 12 - DNA, 45 bp, Artificial Sequence
  Description of Artificial Sequence synthetic primer
ccttttcgctgagcggccgcaatcacaggtaccgtttttgtcgtc

SEQ ID NO: 13 - DNA, 24 bp, Artificial Sequence
  Description of Artificial Sequence synthetic primer
atgcctcaatcctgggaagaactg

SEQ ID NO: 14 - DNA, 24 bp, Artificial Sequence
  Description of Artificial Sequence synthetic primer
cttgacgtagaagacggcaccggc

SEQ ID NO: 15 - DNA, 36 bp, Artificial Sequence
  Description of Artificial Sequence synthetic primer
cccgcagcacatatgcttcacatcctctgtcaaggc

SEQ ID NO: 16 - DNA, 21 bp, Artificial Sequence
  Description of Artificial Sequence synthetic primer
atgacaaacatctcatcaggg

SEQ ID NO: 17 - DNA, 65 bp, Artificial Sequence
  Description of Artificial Sequence synthetic primer
ggggacaagtttgtacaaaaaagcaggcttcgcggccgcgaagcgttagtgaaagggccacggtc

SEQ ID NO: 18 - DNA, 63 bp, Artificial Sequence
  Description of Artificial Sequence synthetic primer
ggggaccactttgtacaagaaagctgggttcgcggccgcaccctgtccatcctgaaagagttg

SEQ ID NO: 19 - DNA, 20 bp, Artificial Sequence
  Description of Artificial Sequence synthetic primer
ggaaactcattggcttggaa

SEQ ID NO: 20 - DNA, 20 bp, Artificial Sequence
  Description of Artificial Sequence synthetic primer
cacccttagcaccacaaggt

SEQ ID NO: 21 - DNA, 53 bp, Artificial Sequence
  Description of Artificial Sequence synthetic primer
ggggacaactttgtatagaaaagttgaaggatgactattccagtgattagcac

SEQ ID NO: 22 - DNA, 27 bp, Artificial Sequence
  Description of Artificial Sequence synthetic primer
gagaagacgaaactcgtgctttgagtg

SEQ ID NO: 23 - DNA, 27 bp, Artificial Sequence
  Description of Artificial Sequence synthetic primer
cactcaaagcacgagtttcgtcttctc

SEQ ID NO: 24 - DNA, 53 bp, Artificial Sequence
  Description of Artificial Sequence synthetic primer
ggggactgcttttttgtacaaacttgaagggagtacttgtgtccacgtcgttg

SEQ ID NO: 25 - DNA, 22 bp, Artificial Sequence
  Description of Artificial Sequence synthetic primer
gtggtaggcggccaggtagaac

SEQ ID NO: 26 - DNA, 22 bp, Artificial Sequence
  Description of Artificial Sequence synthetic primer
cagcatcttcgtggaggtgcgc

SEQ ID NO: 27 - DNA, 59 bp, Artificial Sequence
  Description of Artificial Sequence synthetic primer
ggggacaagtttgtacaaaaaagcaggctaacccgccttccgactacatatccacaatc

SEQ ID NO: 28 - DNA, 56 bp, Artificial Sequence
  Description of Artificial Sequence synthetic primer
ggggaccactttgtacaagaaagctgggtactcaggaatgaatcagatcaacattc

SEQ ID NO: 29 - DNA, 54 bp, Artificial Sequence
  Description of Artificial Sequence synthetic primer
ggggacagctttcttgtacaaagtggaagtatcaggattgatgcctgaaacatc

SEQ ID NO: 30 - DNA, 53 bp, Artificial Sequence
  Description of Artificial Sequence synthetic primer
ggggacaactttgtataataaagttgagatctgctggtagactagagcctgcc

SEQ ID NO: 31 - DNA, 27 bp, Artificial Sequence
  Description of Artificial Sequence synthetic primer
cacaggaatcacagcagaacagtcatc

SEQ ID NO: 32 - DNA, 25 bp, Artificial Sequence
  Description of Artificial Sequence synthetic primer
tcccatttgctgttgatggagcagc

SEQ ID NO: 33 - DNA, 31 bp, Artificial Sequence
  Description of Artificial Sequence synthetic primer
gatctgagatgtcacatgcgtgtagatagac

SEQ ID NO: 34 - DNA, 27 bp, Artificial Sequence
  Description of Artificial Sequence synthetic primer
caattgatcttctctcgtggcaaagag

SEQ ID NO: 35 - DNA, 22 bp, Artificial Sequence
  Description of Artificial Sequence synthetic primer
tggttgcgaaggctgcaaagac

SEQ ID NO: 36 - DNA, 26 bp, Artificial Sequence
  Description of Artificial Sequence synthetic primer
tgtacacgctgacctcgcatatgaag

(19) United States
(12) Patent Application Publication
(10) Pub. No.: US 2009/0233287 A1
(43) Pub. Date: Sep. 17, 2009

(54) PRODUCTION OF COMPOUNDS IN A RECOMBINANT HOST

(76) Inventors: Roelof Ary Lans Bovenberg, Rotterdam (NL); Marco Alexander Van den Berg, Poeldijk (NL); Susanne Hage, Delft (NL); Paul Klaassen, Dordrecht (NL); Bernard Meijrink, Vlaardingen (NL); Lourina Madeleine Raamsdonk, Den Haag (NL)

Correspondence Address: NIXON & VANDERHYE, PC, 901 NORTH GLEBE ROAD, 11TH FLOOR, ARLINGTON, VA, 22203, US

(21) Appl. No.: 12/298,191

(22) PCT Filed: Apr. 25, 2007
(86) PCT No.: PCT/EP07/54045

§ 371 Date: Oct. 23, 2008

(30) Foreign Application Priority Data
Apr. 25, 2006 (EP) 06113096.9
Nov. 16, 2006 (EP) 06124179.0

(51) Int. Cl. C12Q 1/68; C12N 1/19; C12P 1/00; C12P 17/06; C12P 17/04; C12P 7/02; C12P 17/16; C12P 7/42

(52) U.S. Cl. 435/6; 435/254.5; 435/41; 435/125; 435/126; 435/155; 435/118; 435/146

(57) ABSTRACT

The present invention provides a recombinant Penicillium chrysogenum strain characterized in that a gene selected from the list consisting of penDE, pcbAB and pcbC is inactivated and the use of such a strain for the preparation of a compound of interest. Furthermore, it is an object of the present invention to provide a method for the production of a compound of interest in a eukaryotic recombinant microorganism comprising the steps of: (a) Reducing the level of secondary metabolite production in said microorganism with 50-100%; (b) Introducing a heterologous gene into said microorganism

### FIELD OF THE INVENTION

The present invention relates to a host cell derived from an industrial production organism and the use of such a host cell in the production of a compound of interest.

### BACKGROUND OF THE INVENTION

Many pharmaceuticals are derived from natural products. Either the natural product itself is the active pharmaceutical ingredient (API) or one or more conversions are applied to obtain an API. Some examples of the former class are taxol (produced by the yew tree), tacrolimus and rapamycin (both produced by Streptomyces species), epothilon B (produced by Sorangium cellulosum) and penicillin G (produced by Penicillium species). Some examples of the latter class are pravastatin (derived from compactin), simvastatin (derived from lovastatin), caspofungin (derived from pneumocandins), clarithromycin (derived from erythromycin) and the semi-synthetic penicillins and cephalosporins (derived from penicillin G or cephalosporin C).

Most of the above products share a mutual production problem, notably the relatively high production costs due to low supply via the natural production host. In some cases this problem is becoming increasingly important due to increasing world consumption, resulting in very high medicine prices. A good example in this field is the production of taxol where current API prices are over 200,000 USD per kilogram. Due to the very low concentration (0.02%) of taxol in the yew tree, the original source of taxol, many trees are needed to isolate a sufficient amount of product. With the development of a semi-synthetic production route from an intermediate (i.e. baccatin III), which can be found in a somewhat higher concentration in the tree, the production costs of taxol were lowered, but there is still much room for improvement as still enormous numbers of trees are needed to isolate enough of the intermediate to produce taxol. Comparing this to typical production titers of products in microorganisms (ranging from mg/L to g/L fermentation broth) it is no surprise that scientists are actively pursuing production of compounds like taxol in microorganisms as these have several advantages (easy containment, higher production titers, lower product isolation costs, less waste (biomass) material). In the case of taxol this is exemplified by the pursuits of groups like Strobel et al. and Croteau et al., who describe microorganisms capable of taxol production isolated from nature and engineering of Saccharomyces cerevisiae, respectively (see for examples Stierle et al. Science 1993, Apr. 9, 260(5105), 214-216 and De Jong et al. Biotechnol. Bioeng. 2006, Feb. 5, 93(2), 212-224).

Although very promising, there are several drawbacks to these approaches. Isolation of microorganisms from nature capable of producing the API totally depends on the actual existence of such microorganisms. And if they exist, they will mostly produce the API in small amounts, as this is the minimum amount needed by the microorganism to survive in its natural environment. This is the case with all the microorganisms isolated over the years that are capable of producing taxol. Typically, these micro-organisms produce in the μg/L range, which is much too low for an economically viable process. Hence, a lot of effort is needed to enhance the production titers of these species, using technologies like UV-mutagenesis and/or metabolic engineering. Moreover, suitable fermentation media for these microorganisms and/or products need to be developed. All together this would end up in lengthy and expensive research.

Also very promising is the second option, i.e. transferal of the pathway from the natural host to a microorganism. Nevertheless, also this approach has several drawbacks. A pre-requisite for this approach is the availability of all genes encoding the enzymes of the biosynthetic pathway. In the case of taxol still several genes have to be isolated, but there are examples where all genes are available and transferred to other species. For instance, Tang et al. (Science 2000, Jan 28; 287(5453), 640-642) transferred the biosynthetic pathway of epothilon from Sorangium cellulosum to Streptomyces coelicolor. The lack of a satisfactory fermentation process in the first organism leading to economically impractical production triggered the production of epothilones A and B in a ‘fermentation-friendly’ heterologous host. A logical approach, but unfortunately the results are not very promising. While Sorangium cellulosum can produce up to 20 mg/L, the Streptomyces coelicolor transformant produced 50-100 μg/L, which actually is a 20-40-fold lower production titer.

Therefore, an economically feasible way of solving the problem of low API and/or API-building block titers via fermentation is not available and is extremely desirable.

### SUMMARY OF THE INVENTION

The present invention provides a recombinant Penicillium chrysogenum strain characterized in that a gene selected from the list consisting of penDE, pcbAB and pcbC is inactivated and the use of such a strain for the preparation of a compound of interest. Furthermore, it is an object of the present invention to provide a method for the production of a compound of interest in a eukaryotic recombinant microorganism comprising the steps of:
(a) Reducing the level of secondary metabolite production in said microorganism with 50-100%;
(b) Introducing a heterologous gene into said microorganism

### DETAILED DESCRIPTION OF THE INVENTION

The term “Active Pharmaceutical Ingredient” or “API” is defined herein as a molecule which is the active ingredient of a drug.

The term “API-building block” is defined herein as a molecule that can be used in the preparation of an API.

The term “complex medium” or “complex fermentation medium” refers to a fermentation medium comprising lactose (40 g/L), corn steep solids (20 g/L), CaCO₃ (10 g/L), KH₂PO₄ (7 g/L) and phenyl acetic acid (0.5 g/L) having a pH-value of 6.0. Fermenting a microorganism on a complex medium according to the above specifications allows for quantifying fermentation titers within the scope of the present invention. The composition of the complex medium as defined above does not limit the scope of the present invention in itself.

The term “compound of interest” or “COI” comprises any molecule that is not produced in the recombinant organism prior to introduction of one or more heterologous genes or that is produced in the recombinant organism prior to introduction of one or more heterologous genes but only at a level that is at least 50% below the production level after introduction of one or more heterologous genes.

The term “control sequences” is defined herein to include all components, which are necessary or advantageous for the expression of a polypeptide. Each control sequence may be native or foreign to the nucleic acid sequence encoding the polypeptide. Such control sequences may include, but are not limited to, a promoter, a leader, optimal translation initiation sequences (as described in Kozak, 1991, J. Biol. Chem. 266:19867-19870), a secretion signal sequence, a pro-peptide sequence, a polyadenylation sequence, a transcription terminator. At a minimum, the control sequences include a promoter, and transcriptional and translational stop signals. The control sequence may be an appropriate promoter sequence containing transcriptional control sequences. The promoter may be any nucleic acid sequence, which shows transcription regulatory activity in the cell including mutant, truncated, and hybrid promoters, and may be obtained from genes encoding extra cellular or intracellular polypeptides. The promoter may be either homologous or heterologous to the cell or to the polypeptide. The promoter may be derived from the donor species for the gene to be expressed or from any other source. An alternative way to control expression levels in eukaryotes is the use of introns. Higher eukaryotes have genes consisting of exons and introns.

The term “exons” is defined herein to include all components of the Open Reading Frame (ORF), which are translated into the protein.

The term “expression” includes any step involved in the production of a polypeptide and may include transcription, post-transcriptional modification, translation, post-translational modification and secretion.

The term “inactivation” refers to any treatment of a gene resulting in reduction or absence of the gene product as compared to the situation prior to inactivation. Inactivation may for example be the result of deletion, modification, disruption or silencing of the gene and/or its promoter. In the context of the present invention, gene inactivation is carried out through recombinant techniques.

The term “introns” is defined herein to include all components, which are not comprised within the ORF and not translated in the protein.

The term “nucleic acid construct” is synonymous with the term “expression vector” or “cassette” when the nucleic acid construct contains all the control sequences required for expression of a coding sequence in a particular host organism.

The term “Open Reading Frame” is defined herein as a polynucleotide starting with the sequence ATG, the codon for methionine, followed by a consecutive series of codons encoding all possible amino acids and after a certain number interrupted by a termination codon. This Open Reading Frame can be translated into a protein. A polynucleotide containing a gene isolated from the genome is a so-called genomic DNA or gDNA sequence of that gene, including all exons and introns. A polynucleotide containing a gene isolated from mRNA via reverse transcriptase reactions is a so-called copy DNA or cDNA sequence of that gene, including only the exons, while the introns are spliced out through the cells machinery. This latter type of DNA is of particular use when expressing eukaryotic genes of interest in prokaryotic hosts.

The term “operably linked” is defined herein as a configuration in which a control sequence is appropriately placed at a position relative to the coding sequence of the DNA sequence such that the control sequence directs the production of a polypeptide.

The term “recombinant” refers to a method involving a step in which external nucleic acid is added to a host cell. An example may be genetic engineering leading to genetically modified organisms, for instance as is the case with inactivation of penicillin biosynthetic genes.

It is an object of the present invention to provide a method for developing high titer API and/or API-building block production strains and fermentation processes.

In a first aspect of the present invention there is provided a platform strain having at least one of the following characteristics:
Can be used for production of several different product classes
Is suitable for scale-up and large scale production processes
Has a GRAS (Generally Recognized As Safe) status
Does not produce any unwanted compounds (toxins, β-lactams, etc)
Has a good registration track record

In the context of this invention a “platform strain” is defined as a strain that displays at least one of the above characteristics, preferably two of the above characteristics, more preferably three of the above characteristics, even more preferably four of the above characteristics and most preferably all of the above characteristics.

In one embodiment of the first aspect there is provided an example of a platform strain derived from a β-lactam producing microorganism. Preferably this microorganism is a penicillin production strain, Penicillium chrysogenum. More preferably, the penicillin production strain is CBS 455 95. This organism underwent several rounds of classical strain improvement and subsequent process adaptations/improvements over the last 60 years to come towards the current high titer penicillin G fermentation processes. The numerous changes in the DNA of the organism resulted not only in an increased flux and yield towards the product penicillin G (see FIG. 1), but moreover also resulted in morphological changes and adaptations to the harsh conditions in 150,000-liter fermentation vessels (i.e. oxygen limitation, shear forces, glucose limitation and the like). By inactivating or deleting the β-lactam biosynthetic machinery, a platform strain is obtained that is devoid of any β-lactam production capability, but still retains all the mutations that result in the good performance on industrial scale, such as resistance to shear forces, suitability for scaling up, high metabolic flux towards metabolites, adapted to a defined medium, adapted to industrial Down Stream Processing, and low viscosity profile (i.e. morphological, regulatory and metabolic mutations). In the Penicillin chrysogenum strain of the present invention, at least the β-lactam biosynthetic genes pcbC, encoding for isopenicillin N synthase, are inactivated. Accordingly, according to a preferred embodiment, the strain of the invention is a recombinant Penicillium chrysogenum having an inactive pcbC gene. Inactive means the expression of this pcbC gene is reduced to 50% or less, preferably 5% or less, more preferably 2% or less and most preferably less than 0.1%. Said activities can be determined using methods known to the person skilled in the art such as Northern Blot analysis, micro array analysis, rtPCR analysis or the like.

Preferably, also the other β-lactam biosynthetic genes, pcbAB, encoding for L-(α-aminoadipyl)-L-cysteinyl-D-valine synthetase, and/or penDE, encoding for acyl-coenzyme A:isopenicillin N acyltransferase, are inactivated. Accordingly, in a preferred embodiment, the strain of the invention is a recombinant Penicillium chrysogenum having an inactive gene selected from the group consisting of pcbC, pcbAB and penDE. More preferably, all the genes mentioned are inactivated by removal of part of the genes. Most preferred is that the gene sequences are completely removed (complete deletion). As complete removal of these genes leads to Penicillium chrysogenum strains that are devoid of any β-lactam biosynthetic capacity and therefore are very useful strains for producing all sorts of products. According to a most preferred embodiment, the strain of the invention is a recombinant Penicillium chrysogenum strain lacking the gene pcbC and/or pcbAB and/or penDE. Highly suitable examples of deletions or inactivations are those wherein all three said genes are inactivated or deleted but also those wherein only pcbAB or only pcbC are inactivated or deleted. Most preferably said strain is derived from CBS 455 95.

Despite the fact that industrial organisms can be very cumbersome to work with, this Penicillium chrysogenum platform strain is surprisingly well transformable and capable of producing various metabolites at titers much higher than the natural producing hosts of such products. As a result of this, API and/or API-building block producing Penicillium chrysogenum strains are obtained that can be scaled up to an industrial process.

Preferably the platform strain is obtained from an organism capable of producing in an industrial environment. Such organisms typically can be defined as having high productivities and/or high yield of product on amount of carbon source consumed and/or high yield of product on amount of biomass produced and/or high rates of productivity and/or high product titers. Such organisms are extremely useful for conversion into the platform strain of the present invention. For penicillin G producing Penicillium chrysogenum strains for instance, such high titers are titers higher than 1.5 g/L penicillin G, preferably higher than 2 g/L penicillin G, more preferably higher than 3 g/L penicillin G, most preferably higher than 4 g/L penicillin G. The aforementioned values apply to fermentation titers after 96 h in complex fermentation medium. Suitable industrial strains are strains as mentioned in the experimental part (General Methods). According to a most preferred embodiment, the strain of the invention is a recombinant Penicillium chrysogenum strain lacking the gene pcbC and/or pcbAB and/or penDE and is a strain producing more than 1.5 g/L penicillin G after 96 h fermentation on complex medium (prior to removal or inactivation of said genes).

In a preferred embodiment classical strain improvement procedures (i.e. classical mutagenesis and screening) can be used to further improve the characteristics of strains like CBS 455 95 (for a detailed description of such methods see Lein, J., 1986, The Panlabs Penicillium strain improvement program; in: Overproduction of microbial metabolites, Vanek, Z. and Hostalek, Z. (eds.), 105-140, Butterworths, Stoneham, Mass.). By applying these methods Penicillium chrysogenum strains are obtained, which are either better antibiotic producers (much more than 4 g/L) and/or better adapted to industrial fermentation conditions as compared to CBS 455 95. The above procedures can not only be applied to CBS 455 95 or the like, but also to derivatives thereof. Subsequently, β-lactam production is reduced according to the present invention resulting in platform strains. Optionally these platform strains can be further optimized by one or more classical strain improvements.

In another embodiment the platform strain can be even further improved by further minimizing the number of unwanted products. In this respect all non-essential genes, such as genes involved in production of secondary metabolites, or the pathways associated with these unwanted products can be inactivated and/or deleted. This will limit the pathways competing for carbon and thereby further increase the carbon flux towards the product.

The platform strain principle could be applied to other industrial strains of several eukaryotic species, like Aspergillus niger, Aspergillus oryzae, Aspergillus sojae, Aspergillus terreus, Chrysosporium lucknowense, Kluyveromyces lactis, Penicillium brevicompactum, Penicillium citrinum, Pichia ciferrii, Pichia pastoris, Saccharomyces cerevisiae, Trichoderma reesei. All underwent various rounds of classical mutagenesis, followed by screening and selection for improved industrial production characteristics. By removing (i.e. deleting) parts of or complete pathways of unwanted products the strains remain their desired industrial fermentation characteristics and high flux to metabolites (including enzymes). The platform strain principle could also be applied to fungal strains that are very amenable for genetic modifications like Aspergillus nidulans or Neurospora crassa. Said organisms can be quickly adapted to the need for producing large amounts of API's and/or API building blocks. The platform strain principle could even be applied to industrial strains of several prokaryotic species like Streptomyces clavuligerus, Streptomyces avermitilis, Streptomyces peucetius, Corynebacterium glutamicum, Escherichia coli. All underwent various rounds of classical mutagenesis, followed by screening and selection for improved industrial production characteristics. By removing (i.e. deleting) parts of or complete pathways of unwanted products the strains retain their desired industrial fermentation characteristics and high flux to metabolites (including enzymes). Although in U.S. Pat. No. 6,180,366 a method is disclosed in which a polypeptide is produced in a filamentous fungal cell having reduced trichothecene production, this publication only addresses the problem of reducing the production of an unwanted product in a host and not the problem of realizing enhanced production of a compound of interest (COI).

The second aspect of the invention is a method for producing a COI using a eukaryotic recombinant microorganism, for instance the platform strain described above.

Firstly, the platform strain is obtained by reducing the copy number of the relevant biosynthetic genes of the unwanted product pathway from a strain of choice. For example, in case of industrial, high penicillin producing Penicillium chrysogenum, these are the β-lactam biosynthetic genes. Following reduction of the copy number, said genes are inactivated, preferably deleted. All industrial strain lineages of Penicillium chrysogenum underwent numerous rounds of classical strain improvement resulting in three general types of mutations:
(i) Direct amplification of the biosynthetic genes resulting in increased activity of the enzymes of the penicillin metabolite pathway
(ii) Modifications in primary metabolism genes, ultimately resulting in various adapted metabolic rearrangements, all leading to higher flux towards the end product. Examples: increased synthesis of amino acid building blocks, decreased consumption of phenyl acetic acid and the like.
(iii) Cell structure modifications, resulting in alteration of morphology, membrane composition, organelles organization and thereby ‘facilitating’ high metabolic fluxes and fermentation at industrial scale. Examples: increased numbers of peroxisomes, which are one of the ‘assembly lines’ of penicillin synthesis.

There is a significant distinction on DNA level in the type of mutations of class (i) as compared to classes (ii) and (iii). While the latter two classes are mostly isolated mutations, deletions, duplications and/or alterations on base pair level, the mutation in class (i) is a very distinct amplification of a 60 to 100 kb region, resulting in several direct and inverted repeats on the genome. This might lead to a significant genetic instability, resulting in an instable and changing population. In fact this means that in a given penicillin production strain all mutations of class (ii) and (iii) are fixed, but the exact copy number of the mutation of class (i) can fluctuate. Using this principle and techniques known to the ones skilled in the art, stable isolates can be obtained where only one copy of the penicillin biosynthetic genes is still present. A preferred method is:
(i) Isolating protoplasts from suitable Penicillium chrysogenum strains,
(ii) Plating these protoplasts on regenerating agar plates,
(iii) Incubating the agar plates until colonies are visible,
(iv) Determining the β-lactam productivity of the colonies using assays such as bioassays, HPLC assays, enzymatic assays, colorimetric assays, NMR assays and the like,
(v) Selecting colonies with reduced β-lactam productivity as compared to the parent strain.

Depending on the copy number of the starting strain this situation can be obtained in one, two, three or several rounds of screening and selection. For this specific characteristic the isolate is then comparable to the type strain of the species, NRRL1951, and its first descendants after classical strain improvement, up to Wisconsin 54-1255, all of which contain one copy of the penicillin biosynthetic genes. The major difference is that the one-copy isolate derived from the high producing strain still contains all the other mutations of class (ii) and (iii) making it an industrial high producing strain as compared to the strains from NRRL1951 to Wisconsin 54-1255. Subsequently, the last set of penicillin biosynthetic genes can be deactivated, preferably deleted, using state-of-the-art recombination techniques. A detailed overview of these steps is given in the examples and summarized in the following steps:
(a) Isolating an isolate with a single genomic copy of the penicillin gene cluster from a Penicillium strain
(b) Inactivating, preferably deleting gene pcbC from the isolate obtained in step (a)
(c) Optionally inactivating, preferably deleting genes pcbAB and/or penDE from the isolate obtained in steps (a) or (b)

The genes can be partly inactivated. Accordingly, according to a preferred embodiment, the strain of the invention is a recombinant Penicillium chrysogenum having an inactive pcbC gene. Inactive means the expression of this pcbC gene is reduced to 50% or less, preferably 5% or less, more preferably 2% or less and most preferably less than 0.1%. Said activities can be determined using methods known to the person skilled in the art such as Northern Blot analysis, micro array analysis, rtPCR analysis or the like. More preferably, the gene sequences are completely removed. As complete removal of these genes leads to Penicillium chrysogenum strains that are devoid of any β-lactam biosynthetic capacity and therefore are very useful strains for producing all sorts of products. Recombination techniques that can be applied are well known for the ones trained in the art (i.e. Single Cross Over or Double Homologous Recombination).

A preferred strategy for the deletion of one of the mentioned genes (and the replacement) is the gene replacement technique described in EP 357,127. The specific deletion of a gene and/or promoter sequence is preferably performed using the amdS gene as selection marker gene as described in EP 635,574. By means of counter selection on fluoroacetamide media as described in EP 635,574, the resulting strain is selection marker free and can be used for further gene modifications. Alternatively or in combination with other mentioned techniques, a technique based on in vivo recombination of cosmids in Escherichia coli can be used, as described in: A rapid method for efficient gene replacement in the filamentous fungus Aspergillus nidulans (2000) Chaveroche, M-K. et al., Nucleic Acids Research, vol. 28, no. 22. This technique is applicable to filamentous fungi other than Penicillium chrysogenum, such as Aspergillus nidulans sterigmatocystin mutants, Aspergillus niger nigragillin mutants or Penicillium chrysogenum chrysogenin mutants. Also, the same principle for removing amplified genome fragments can be applied to other industrial production species in which classical strain improvement programs have induced gene and genome duplications. Also, here additional mutations of class (ii) and (iii) are fixed and make sure that the platform strains can thrive in industrial fermentation processes.

Secondly, the platform strain as described above is transformed with a gene or set of genes encoding complete pathways towards compounds of interest. Therefore, the platform strain of the present invention can be used for the preparation of a COI. This can be, but is not limited to, API's or API-building blocks, obtained from the natural producing species.

One embodiment describes the retransformation of the platform strain with the three penicillin biosynthetic genes (i.e. pcbAB, pcbC and penDE encoding the enzymes L-aminoadipyl)-L-cysteinyl-D-valine synthase, isopenicillin N synthase and iso-penicillin N:acyl CoA acyltransferase, respectively). As outlined in the experimental section, it was demonstrated that transformants regain their capability of penicillin G synthesis.

In a preferred embodiment the COI is compactin. Thereto some genes involved in the compactin synthesis pathway are introduced into the platform strain of the first aspect. Preferably, the platform strain is transformed with a set of nine genes (mlcA, mlcB, mlcC, mlcD, mlcE, mlcF, mlcG, mlcH and mlcR), including putative transporters and transcriptional regulators, as outlined in detail in the experimental section.

The scope of this invention is not limited to the examples of compounds of interest and examples of platform strains given. These examples are given to illustrate the applicability of a platform strain to several compounds of interest and to several platform strains. Theoretically, all eukaryotic gene sets can be expressed in a platform strain. Compounds of interest produced by these gene sets may be secondary metabolites such as alkaloids, coumarin, flavonoid, polyketide, quinine, steroid, peptide, or terpene. The secondary metabolite may be an antibiotic, bacteriocide, fungicide, hormone, insecticide, or rodenticide. Preferred compounds of interest are antibiotics, aflatoxin, aphidicolin, compactin, ergotamine, fumonisin, lovastatin, lysergic acid, paxicillin and trichothecene. Other compounds of interest produced by these gene sets may be primary metabolites such as amino acids, citric acid, fatty acids, nucleosides, nucleotides, polyols such as mannitol and sorbitol, succinic acid, sugars, triglycerides, or vitamin. Preferred primary metabolites are butyric acid, citric acid, ethanol and succinic acid.

In yet another embodiment the production of these compounds of interest in the platform strain may be improved by using proteins with improved kinetic features. These can be homologous proteins involved in the biosynthesis of said compounds of interest. Such a “homologue” or “homologous sequence” is defined as a DNA sequence encoding a polypeptide that displays at least one activity of the polypeptide encoded by the original DNA sequence isolated from the species naturally producing the API and/or API building block (i.e. for compactin this is Penicillium citrinum). Such a polypeptide has an amino acid sequence which is at least 40% identical to the amino acid sequence of the protein encoded by the specified DNA sequence. Using this approach various advantages are obtained such as to overcome feedback inhibition, improvement of secretion and reduction of byproduct formation. A homologous sequence may encompass polymorphisms that may exist in cells from different populations or within a population due to natural allelic or intra-strain variation. A homologue may further be derived from a species other than the species where the specified DNA sequence originates from, or may be artificially designed and synthesized. DNA sequences related to the specified DNA sequences and obtained by degeneration of the genetic code are also part of the invention.

The nucleic acid constructs of the present invention, e.g. expression constructs, contain at least one gene of interest (used for the production of the COI), but in general contain several genes of interest; each operably linked to one or more control sequences, which direct the expression of the encoded COI in the platform strain. The nucleic acid constructs may be supplied to the platform strain as one polynucleotide or as several polynucleotides. Also these nucleic acid constructs may be integrated at one chromosomal locus or at several chromosomal loci. To obtain the highest possible productivity a balanced expression of all genes of interests is crucial. Therefore, a range of promoters can be useful. Preferred promoters for application filamentous fungal cells like Penicillium chrysogenum are known in the art and can be, for example, the promoters of the gene(s) derived from the natural producers of the API and/or API-building block; the glucose-6-phosphate dehydrogenase gpdA promoters; the Penicillium chrysogenum pcbAB, pcbC and penDE promoters; protease promoters such as pepA, pepB, pepC; the glucoamylase glaA promoters; amylase amyA, amyB promoters; the catalase catR or catA promoters; the glucose oxidase goxC promoter; the beta-galactosidase lacA promoter; the α-glucosidase ag/A promoter; the translation elongation factor tefA promoter; xylanase promoters such as xlnA, xlnB, xlnC, xlnD; cellulase promoters such as eglA, eglB, cbhA; promoters of transcriptional regulators such as areA, creA, xlnR, pacC, prtT, alcR, or any other. Said promoters can easily be found by the skilled person, amongst others, at the NCBI Internet website (http://wwwcbi.nim.nih.gov/entez/). In case of platform strains derived from other than filamentous fungal species the choice of promoters will be determined by the choice of the host.

In a preferred embodiment, the promoter may be derived from a gene, which is highly expressed (defined herein as the mRNA concentration with at least 0.5% (w/w) of the total cellular mRNA). In another preferred embodiment, the promoter may be derived from a gene, which is medium expressed (defined herein as the mRNA concentration with at least 0.01% until 0.5% (w/w) of the total cellular mRNA). In another preferred embodiment, the promoter may be derived from a gene, which is low expressed (defined herein as the mRNA concentration lower than 0.01% (w/w) of the total cellular mRNA).

In a still more preferred embodiment micro array data is used to select genes, and thus promoters of those genes, that have a certain transcriptional level and regulation. In this way one can adapt the gene expression cassettes optimally to the conditions it should function in. These promoter fragments can be derived from many sources, i.e. different species, PCR amplified, synthetically and the like.

The control sequence may also include a suitable transcription termination sequence, a sequence recognized by a eukaryotic cell to terminate transcription. The terminator sequence is operably linked to the 3′-terminus of the nucleic acid sequence encoding the polypeptide. Any terminator, which is functional in the cell, may be used in the present invention.

Preferred terminators for filamentous fungal cells are obtained from the genes encoding Aspergillus oryzae TAKA amylase; the Penicillium chrysogenum pcbAB, pcbC and penDE terminators; Aspergillus niger glucoamylase; Aspergillus nidulans anthranilate synthase; Aspergillus niger alpha-glucosidase; Aspergillus nidulans trpC gene; Aspergillus nidulans amdS; Aspergillus nidulans gpdA; Fusarium oxysporum trypsin-like protease. Even more preferred terminators are the ones of the gene(s) derived from the natural producers of the API and/or API-building block. In case of platform strains derived from other than filamentous fungal species the choice of termination sequences will be determined by the choice of the host.

The control sequence may also be a suitable leader sequence, a non-translated region of an mRNA that is important for translation by the cell. The leader sequence is operably linked to the 5′-terminus of the nucleic acid sequence encoding the polypeptide. Any leader sequence, which is functional in the cell, may be used in the present invention. Preferred leaders for filamentous fungal cells are obtained from the genes encoding Aspergillus oryzae TAKA amylase and Aspergillus nidulans triose phosphate isomerase and Aspergillus niger glaA.

The control sequence may also be a polyadenylation sequence, a sequence which is operably linked to the 3′-terminus of the nucleic acid sequence and which, when transcribed, is recognized by the filamentous fungal cell as a signal to add polyadenosine residues to transcribed mRNA. Any polyadenylation sequence, which is functional in the cell, may be used in the present invention.

Preferred polyadenylation sequences for filamentous fungal cells are obtained from the genes encoding Aspergillus oryzae TAKA amylase, Aspergillus niger glucoamylase, Aspergillus nidulans anthranilate synthase, Fusarium oxysporum trypsin-like protease and Aspergillus niger alpha-glucosidase.

Control sequences may be the Kozak sequences, coding translation initiation sequences and termination sequences such as described in WO 2006/077258.

For a polypeptide to be secreted, the control sequence may also include a signal peptide-encoding region, coding for an amino acid sequence linked to the amino terminus of the polypeptide, which can direct the encoded polypeptide into the cell's secretory pathway. The 5′-end of the coding sequence of the nucleic acid sequence may inherently contain a signal peptide-coding region naturally linked in translation reading frame with the segment of the coding region, which encodes the secreted polypeptide. Alternatively, the 5′-end of the coding sequence may contain a signal peptide-coding region, which is foreign to the coding sequence. The foreign signal peptide-coding region may be required where the coding sequence does not normally contain a signal peptide-coding region. Alternatively, the foreign signal peptide-coding region may simply replace the natural signal peptide-coding region in order to obtain enhanced secretion of the polypeptide such as for instance described in WO 90/15860.

In case of eukaryotic platform strains the control sequence may include organelle targeting signals. Such a sequence is encoded by an amino acid sequence linked to the polypeptide, which can direct the final destination (i.e. compartment or organelle) within the cell. The 5′- or 3′-end of the coding sequence of the nucleic acid sequence may inherently contain these targeting signals coding region naturally linked in translation reading frame with the segment of the coding region, which encodes the polypeptide. The various sequences are well known to the persons trained in the art and can be used to target proteins to compartments like mitochondria, peroxisomes, endoplasmatic reticulum, golgi apparatus, vacuole, nucleus and the like.

The nucleic acid construct may be an expression vector. The expression vector may be any vector (e.g. a plasmid or virus), which can be conveniently subjected to recombinant DNA procedures and can bring about the expression of the nucleic acid sequence encoding the polypeptide. The choice of the vector will typically depend on the compatibility of the vector with the cell into which the vector is to be introduced. The vectors may be linear or closed circular plasmids.

The vector may be an autonomously replicating vector, i.e. a vector, which exists as an extra chromosomal entity, the replication of which is independent of chromosomal replication, e.g. a plasmid, an extra chromosomal element, a mini chromosome, or an artificial chromosome. An autonomously maintained cloning vector for a filamentous fungus may comprise the AMA1-sequence (see e.g. Aleksenko and Clutterbuck (1997), Fungal Genet. Biol. 21: 373-397).

Alternatively, the vector may be one which, when introduced into the cell, is integrated into the genome and replicated together with the chromosome(s) into which it has been integrated. The integrative cloning vector may integrate at random or at a predetermined target locus in the chromosomes of the host cell. In a preferred embodiment of the invention, the integrative cloning vector comprises a DNA fragment, which is homologous to a DNA sequence in a predetermined target locus in the genome of host cell for targeting the integration of the cloning vector to this predetermined locus. Preferred target loci in this context can be loci that are not part of a functional gene (i.e. intergenic regions or pseudogenes); loci that are not essential for the fermentation process (i.e. the niaD gene of Penicillium chrysogenum, encoding nitrate reductase); loci that give rise to high expression (i.e. as described in EP 357127). In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is at least 30 bp, preferably at least 0.1 kb, more preferably at least 0.2 kb, still more preferably at least 0.5 kb, even more preferably at least 1 kb, most preferably at least 2 kb.

The efficiency of targeted integration of a nucleic acid construct into the genome of the host cell by homologous recombination, i.e. integration in a predetermined target locus, is preferably increased by augmented homologous recombination abilities of the host cell. Such phenotype of the cell preferably involves a deficient hdfA or hdfB gene as described in WO 05/95624. WO 05/95624 discloses a preferred method to obtain a filamentous fungal cell comprising increased efficiency of targeted integration.

The vector system may be a single vector or plasmid or two or more vectors or plasmids, which together contain the total DNA to be introduced into the genome of the host cell. However in the present invention the constructs are preferably integrated in the genome of the host strain. As this is a random process this even can result in integration in genomic loci, which are highly suitable to drive gene expression, resulting in high amounts of enzyme and subsequently in high productivity.

Fungal cells may be transformed using protoplasts. Suitable procedures for transformation of fungal host cells are described in EP 238.023 and Yelton et al., 1984, Proceedings of the National Academy of Sciences USA 81, 1470-1474. Suitable procedures for transformation of filamentous fungal host cells using Agrobacterium tumefaciens are described by de Groot M. J. et al. (1998, Nat. Biotechnol. 16:839-842. Erratum in: Nat. Biotechnol. 1998 16, 1074). Other methods like electroporation, described for Neurospora crassa, may also be applied.

Fungal cells are transformed using co-transformation, i.e. along with gene(s) of interest also a selectable marker gene is transformed. This can be either physically linked to the gene of interest (i.e. on a plasmid) or on a separate fragment. Following transformation, transformants are screened for the presence of this selection marker gene and subsequently analyzed for the presence of the gene(s) of interest. A selectable marker is a product, which provides resistance against a biocide or virus, resistance to heavy metals, prototrophy to auxotrophs and the like. Useful selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC or sutB (sulfate adenyltransferase), trpC (anthranilate synthase), ble (phleomycin resistance protein), as well as equivalents thereof.

In a third aspect of the invention, the platform strain of the first aspect is ideally suitable for deciphering the biological function of genes or (clustered) gene sets and identifying new pharmaceutical products with new applications. There are several methods available for isolating these genes or (clustered) gene sets, all known to the ones trained in the art. Examples of these methods are: isolated random via shotgun cloning, isolated organized via Bacterial Artificial Chromosome (i.e. BAC) libraries or via genome-sequencing projects. In the latter many gene sequences are generated, but it is not possible to assign functions to all genes. Functional determination is also not always possible, due to the lack of resources, good expression systems or molecular tools for the sequenced species. In particular this is true for gene pools that can be mined for pharmaceuticals with improved applications, such as deep see samples, tropical rain forest samples, extremophiles and the like. One way to go forward is to identify in silico all members of gene families that are typically associated with secondary metabolite formation: non-ribosomal peptide synthetases and polyketide systems. As the wild type level of products generated by these systems is generally low, it will be difficult to identify the products from these pathways, let alone to isolate material for clinical trials. These problems can be overcome by expressing these genes and their close neighbors on the genome in the platform strain. In this background expression can be controlled, gene copy number can be controlled and high product titers can be obtained, all facilitating in identifying the products and the function of genes.

### LEGENDS TO THE FIGURES

FIG. 1 is a schematic representation of the invention. Wild type (WT) strains have a fixed ratio to split the incoming carbon over growth, product (penicillin G) and maintenance. In industrial strains this balance is shifted towards product. In the platform strain the penicillin G pathway is removed, so the carbon flux is rebalanced between growth and maintenance. In the new product strain, the industrial carbon flux balance is restored by introducing a new product pathway. Legend: I=wild type Penicillium chrysogenum strain; II=Industrial Penicillium chrysogenum strain; III=Penicillium chrysogenum platform strain; IV=Penicillium chrysogenum platform strain producing a new product; S=carbon source (i.e. sugar); G=penicillin G; X=biomass; M=Maintenance; P=new product (i.e. API building-block or API).

FIG. 2 shows a Southern blot analysis of industrial Penicillium chrysogenum isolates with a single copy of the penicillin biosynthetic gene cluster. Legend: #=isolate number; N=npe10; W=Wis54-1255; I=intermediate parent; N=niaA gene fragment; P=pcbC gene fragment.

FIG. 3 shows the relative penicillin V titers of various strains grown in shake flasks on mineral media with phenoxy acetic acid. On the Y-axis the percentage of penicillin V is given (level of the industrial parent set at 100). Legend: C=industrial parent; I=intermediate parent; W=Wis54-1255; N=npe10; #=isolate number.

FIG. 4 is a representation of the deletion strategy to remove the last copy of the penicillin gene cluster from isolates of industrial Penicillium chrysogenum strains. Legend: A=pcbAB gene; B=pcbC gene; C=penDE gene; M=amdS gene cassette; 3=3 kb flank length; 5=5 kb flank length; 7=7 kb flank length. The hatched areas indicate the homologous flanking regions; diagonal hatches indicate the left flanking and standing hatches indicate the right flanking.

FIG. 5 shows relative penicillin G titers of various strains grown in shake flasks on mineral media with phenyl acetic acid. On the Y-axis the percentage of penicillin G is given with the level of the industrial parent set at 100. Legend: C=industrial parent; I=intermediate parent; W=Wis54-1255; N=npe10; #=isolate number.

FIG. 6 is a schematic representation of the vector containing the three penicillin biosynthetic genes, pDONR221-Pcpencluster. Legend: kan=kanamycin resistance gene; pcbAB=gene encoding L-aminoadipyl)-L-cysteinyl-D-valine synthase; pcbC=gene encoding isopenicillin N synthase; penDE=gene encoding isopenicillin N:acyl CoA acyltransferase,

FIG. 7 is Southern Blot analysis of the P. chrysogenum strains with randomly reintegrated penicillin gene clusters. As a probe, a DNA fragment from the pcbAB terminator region was employed. Legend: M=kb marker DNA; P=Industrial production strain with multiple penicillin gene amplicons; 1=Intermediate parent (i.e. strain with 1 penicillin gene amplicon); 0=Penicillium chrysogenum platform strain; C1-C13=strains with randomly integrated penicillin gene cluster fragments.

FIG. 8 is a schematic representation of the compactin gene cluster (length is 38231 bp). The dashed arrows indicate the genes and there orientation. The small solid arrows indicate the position of the PCR primers used in the cloning strategy. The sizes on top indicate the length of the fragments amplified via PCR (the 10 and 8 kb fragment are combined via several cloning steps to one 18 kb fragment, see examples for details). Legend: A=mlcA gene; B=mlcB gene; C=mlcC gene; D=mlcD gene; E=mlcE gene; F=mlcF gene; G=mlcG gene; H=mlcH gene; R=mlcR gene.

FIG. 9 shows the PCR amplification of the middle part (14.3 kb) and right part (6 kb) of the compactin gene cluster.

FIG. 10 is a schematic overview of cloning strategy for 18 kb left part of the compactin cluster. Panel A: PCR amplification of 10 kb and 8 kb fragments cloned in pCR2.1 TOPO T/A. Panel B: Fusion-cloning of 10 and 8 kb fragments. NotI-SpeI digestion of 8 kb fragment, ligated in NotI-XbaI digested 10 kb plasmid. PCR amplification of internal 6 kb fragment to restore micA open reading frame. Panel C: Final 18 kb fragment transferred via Gateway reaction to pDONR41Zeo.

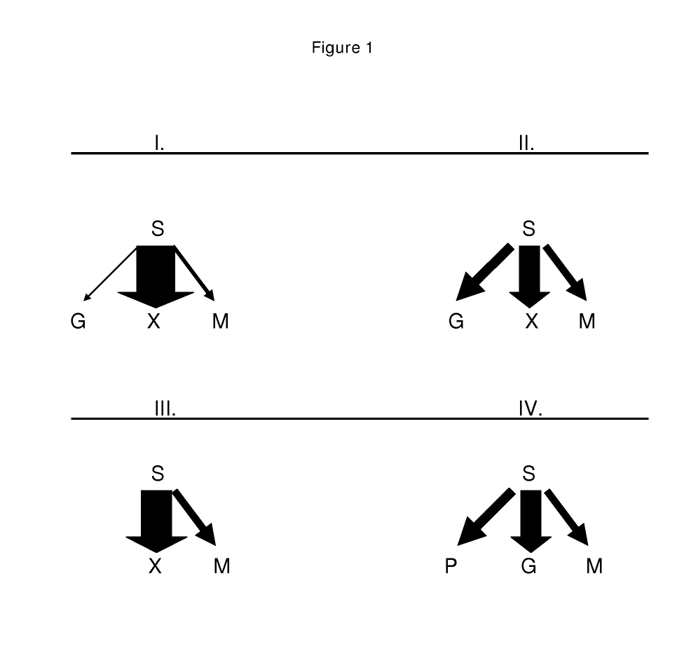

### Legend: CGC=Compactin Gene Cluster; N=NotI; A=Acc651; X=XbaI; S=SpeI.

FIG. 11 depicts two HPLC analyses of the supernatant of fermentation broth. Panel A is from the Penicillium chrysogenum strain deprived of all penicillin biosynthetic gene clusters (i.e. the platform strain). Panel B is from one of the transformants of the Penicillium chrysogenum platform strain with the compactin gene cluster integrated. A peak corresponding to ML-236-A is visible at 2.612 minutes.

### EXAMPLES

### General Methods

Standard DNA procedures were carried out as described elsewhere (Sambrook, J. et al., 1989, Molecular cloning: a laboratory manual, 2^{nd} Ed., Cold Spring Harbor Laboratory Press, Cold Spring Harbor, N.Y.). If specific DNA methods were applied these are specified. DNA was amplified using the proofreading enzyme Herculase polymerase (Stratagene). Restriction enzymes were from Invitrogen or New England Biolabs. As starting strain any industrial Penicillium chrysogenum strain that underwent several rounds of (classical) strain improvement can be used. Examples are: CBS 455.95 (Gouka, R. J. et al., 1991, J. Biotechnol. 20, 189-200); Panlabs P2 (Lein, J., 1986, in ‘Overproduction of microbial metabolites’, Vanek, Z. et al. (eds.), 105-140; Butterworths, Stoneham, Mass.); E1 and AS-P-78 (Fierro, F. et al., 1995, Proc. Natl. Acad. Sci. 92, 6200-6204); BW1890 and BW1901 (Newbert, R. W. et al., 1997, J. Ind. Microbiol. 19, 18-27).

### Example 1

Isolation of a Penicillium chrysogenum platform strain, i.e. a β-lactam free isolate To isolate the platform strain all gene copies encoding β-lactam biosynthetic proteins of an industrial Penicillium chrysogenum strain must be deleted. As these genes are amplified to multiple copies in the industrial Penicillium chrysogenum strain lineages this is not feasible via single gene deletion. The best approach is to first isolate a species of the strain with only one copy of the biosynthetic genes. As all gene amplifications are in direct repeats on the same chromosome (Fierro, F. et al. 1995, Proc. Natl. Acad. Sci. USA 92, 6200-6204), there can be recombinations between different repeats resulting in loss of copies (Newbert, R. W. et al., 1997, J. Ind. Microbiol. 19, 18-27). This is a random process and can be induced via a mutagenic treatment. Isolates should be screened for reduced penicillin production and reduced copy number of the penicillin biosynthetic genes. If enough isolates are screened one is able to find these single copy isolates. That isolate is then used for targeted gene-knockout via homologous recombination.

### Isolation of a Single Copy Isolate

Preparation of Penicillium chrysogenum protoplasts was performed as described in Cantoral, J. M. et al., 1987, Bio/Technol. 5, 494-497, using glucanex instead of novozyme as lysing enzyme. Protoplasts were separated from the mycelium, washed and plated on mineral medium agar (US 2002/0039758), without phenyl acetic acid but supplemented with 15 g/l agar to solidify and 1 M saccharose for osmotic stabilization. Regenerating colonies were transferred to plates without saccharose to induce sporulation. Spores were collected, washed with 0.9 mM NaCl, diluted and plated out on YEPD agar plates (10 g/l Yeast Extract, 10 g/l Peptone, 20 g/l glucose and 15 g/l agar). Isolated colonies were transferred to mineral medium agar plates serving as stock culture plates. 27 random isolates were selected for Southern blot analyses to determine the relative gene-copy number. For this, cells were grown in liquid mineral medium for 48 h at 25° C. and 280 rpm. Cells were harvested, washed with 0.9 mM NaCl and the pellet was frozen in liquid N₂. The frozen cells were grinded using a pestle-and-mortar, transferred to a plastic tube and an equal volume of phenol:CHCl₃:isoamylalcohol (25:24:1) was added. This mixture was vortexed vigorously, centrifuged and the aqueous phase was transferred to a fresh tube. This was repeated twice each time using a fresh volume of phenol:CHCl₃:isoamylalcohol (25:24:1). Finally, DNA was isolated from the aqueous phase by ethanol precipitation according to standard DNA procedures. DNA (3 μg) was digested with EcoRI, separated on 0.6% agarose and transferred to a nylon membrane by Southern Blotting. As probes pcbC and niaA were applied. The former is representative for the copy number of penicillin biosynthetic genes and the latter is an internal control (gene encodes for nitrite reductase) present as single copy in Penicillium chrysogenum strains. The probe sequences were amplified using gene specific primers (Table 1) and labeled with the ECL non-radioactive hybridization kit (Amersham) according to the suppliers instructions. The ratio between the intensity of both signals (pcbC:niaA) was used to estimate the relative gene copy number of the penicillin gene cluster. The parent strain and the single-copy lab strain Wisconsin 54-1255 were applied as controls.

TABLE 1
Primer sequences used to amplify probe sequences

| Gene Target | Forward primer | Reverse primer |
|---|---|---|
| PcbC | SEQ ID NO 1 | SEQ ID NO 2 |
| NiaA | SEQ ID NO 3 | SEQ ID NO 4 |

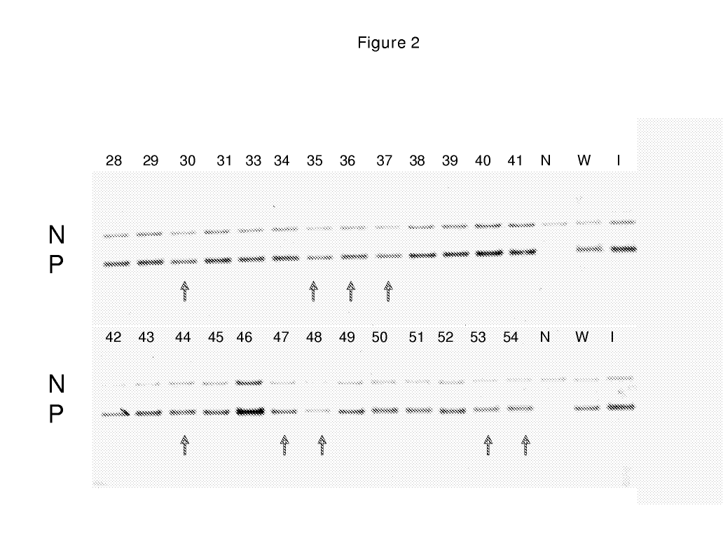

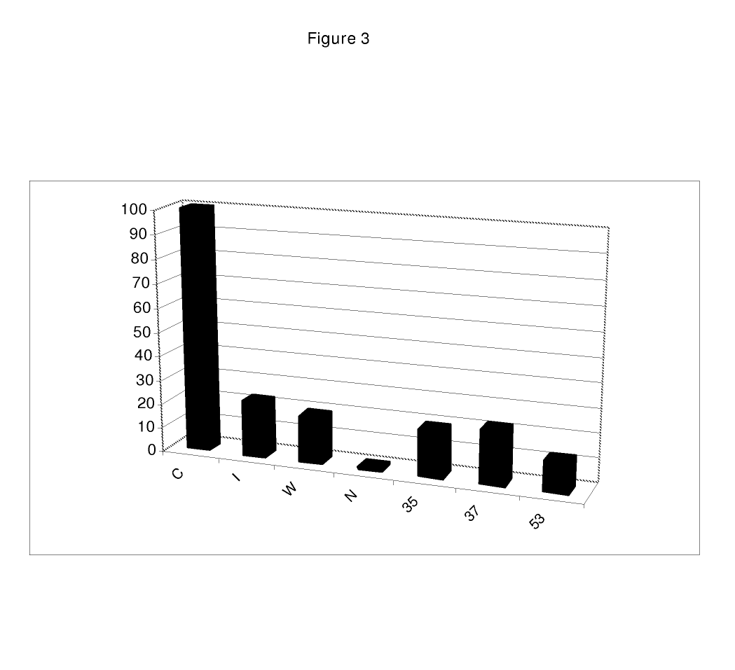

Strains with the lowest ratio were tested for penicillin production in liquid mineral medium with phenoxy acetic acid. Penicillin V production was determined with NMR. All strains with reduced pcbC:niaA ratio's also showed reduced penicillin V titers. One strain was selected that underwent a second round of protoplastation, screening and analyses, identical as described above. Again 27 random isolates were selected and analyzed in detail. From the Southern Blot (see FIG. 2) several putative ‘single copy’ penicillin biosynthetic gene cluster candidates (indicated by the arrows) were identified as these showed a comparable pcbC:niaA ratio as the lab strain Wisconsin 54-1255. Three of these were selected and tested in shake-flasks for penicillin V production. As controls the original industrial Penicillium chrysogenum parent, the intermediate parent, the lab strain Wisconsin 54-1255 and a non-producing isolate of this lab-strain, npe10 (Cantoral, J. M., Gutiérrez, S., Fierro, F., Gil-Espinosa, S., Van Liempt, H., and Martin, J. F., 1993, J. Biol. Chem. 268, 737-744) was used. All three isolates showed a drastic reduced penicillin V titer and comparable to the single-copy lab strain Wisconsin 54-1255, therefore it was concluded that these three isolates are industrial single copy isolates, retaining only one copy of the penicillin gene cluster but also all mutations introduced via classical strain improvement (FIG. 3).

### Deletion Last Copy Penicillin Biosynthetic Genes

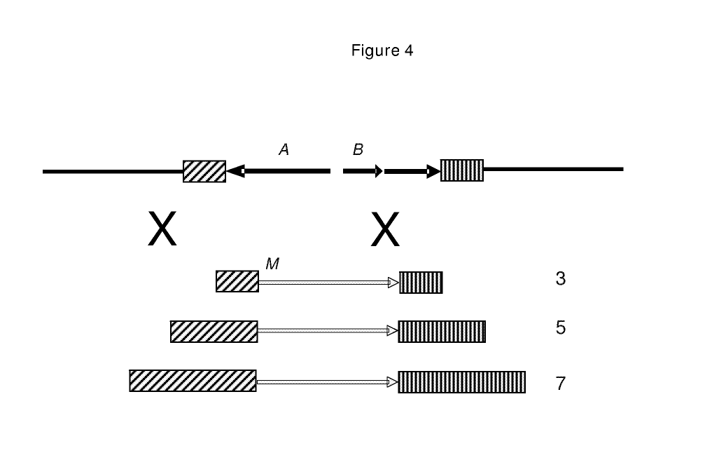

To inactivate the three biosynthetic genes of the last retained copy of the penicillin gene cluster, the double homologous recombination strategy was applied. For this, sequences adjacent to the three biosynthetic genes were used as flankings to target the amdS selection marker to this locus. If double homologous crossover would occur the transformants would be able to use acetamide as the sole carbon source (due to the presence of the amdS gene), should not produce any penicillins and should not hybridize to the pcbC probe. As double homologous crossover is a rare event in Penicillium chrysogenum three constructs were produced: one with 3 kb flanks on either side of the amdS gene, one with 5 kb and one with 7 kb flanks (see FIG. 4). The oligonucleotides applied are listed in Table 2. Following PCR amplification the fragments were cloned in pCRXL via TOPO T/A cloning (Invitrogen). Subsequently all three left flankings (3, 5 and 7 kb) were digested with Acc651 and NotI followed by ligation in pBluescript II SK+ (Invitrogen) pre-digested Acc651 and NotI. The obtained left-flanking plasmids were digested with NotI to facilitate cloning of the right flanks, which were pre-digested with NotI and Eco521. The obtained 3, 5 and 7 kb flanking-plasmids all had a unique NotI site between the left and right flanks, which was used to clone the amdS gene as selection marker. This was obtained by digesting pHELY-A1 (described in WO 04/106347) with NotI and isolating the 3.1 kb PgpdA-AnamdS expression cassette. The thus obtained deletion fragments were isolated following digestion with KpnI and transformed to the penicillin gene cluster single copy isolates. Transformants were selected on their ability to grow on acetamide selection plates and afterwards screened for antibiotic production by replica plating the colonies on mineral medium and overlaying them after 4 days of growth with α-lactam sensitive indicator organism, Escherichia coli strain ESS. If colonies still produced β-lactams this inhibits the growth of the Escherichia coli. 22 out of the 27,076 transformants tested gave no inhibition zone (0.08%) and were selected for further analyses. These 22 isolates were analyzed via colony PCR with three primer sets: niaA, as an internal control for a single copy gene; amdS, for the selection marker; penDE, as indicator for the presence or absence of the penicillin biosynthetic genes.

TABLE 2
Primer sets used for construction double homologous crossover
cassettes. Restriction sites are underlined.

| | | Forward primer | | Reverse primer | |
|---|---|---|---|---|---|
| | | | Restriction | | Restriction |
| | Size | | enzyme | | enzyme |
| Flank | (kb) | Sequence | introduced | Sequence | introduced |
| Left | 7 | SEQ ID NO 5 | Acc65I | SEQ ID NO 6 | NotI |
| Left | 5 | SEQ ID NO 7 | Acc65I | SEQ ID NO 6 | NotI |
| Left | 3 | SEQ ID NO 8 | Acc65I | SEQ ID NO 6 | NotI |
| Right | 3 | SEQ ID NO 9 | NotI | SEQ ID NO 10 | Eco52I, |
| | | | | | Acc65I |
| Right | 5 | SEQ ID NO 9 | NotI | SEQ ID NO 11 | Eco52I, |
| | | | | | Acc65I |
| Right | 7 | SEQ ID NO 9 | NotI | SEQ ID NO 12 | Eco52I, |
| | | | | | Acc65I |

TABLE 3
Primer sequences used to for colony PCR

| Gene | FWD primer | REV primer | Fragment size (bp) |
|---|---|---|---|
| NiaA | SEQ ID NO 3 | SEQ ID NO 4 | 251 |
| AmdS | SEQ ID NO 13 | SEQ ID NO 14 | 653 |
| penDE | SEQ ID NO 15 | SEQ ID NO 16 | 1000 |

All 22 putative mutants gave no signal for the gene penDE, encoding acyltransferase, catalyzing the last step in the penicillin biosynthesis. Two mutants gave no signal for amdS, suggesting that these spontaneously lost the selection marker gene. It was concluded that all 22 isolates are isolates of industrial Penicillium chrysogenum strains without β-lactam biosynthetic genes and qualify for the so-called platform strain.

TABLE 4
Colony PCR on putative Penicillium chrysogenum
platform strain isolates

| | Fragment used for | | | |
|---|---|---|---|---|
| Strain | deletion with | niaA | amdS | penDE |
| Npe10 | — | + | − | − |
| Wisconsin54-1255 | — | + | − | + |
| CBS 455.95 | — | + | − | + |
| Deletion mutant | 3 kb flanks | + | + | − |
| Deletion mutant | 3 kb flanks | + | + | − |
| Deletion mutant | 5 kb flanks | + | + | − |
| Deletion mutant | 5 kb flanks | + | + | − |
| Deletion mutant | 5 kb flanks | + | + | − |
| Deletion mutant | 5 kb flanks | + | + | − |
| Deletion mutant | 7 kb flanks | + | + | − |
| Deletion mutant | 7 kb flanks | + | + | − |
| Deletion mutant | 7 kb flanks | + | + | − |
| Deletion mutant | 7 kb flanks | + | − | − |
| Deletion mutant | 3 kb flanks | + | + | − |
| Deletion mutant | 3 kb flanks | + | + | − |
| Deletion mutant | 3 kb flanks | + | + | − |
| Deletion mutant | 5 kb flanks | + | + | − |
| Deletion mutant | 5 kb flanks | + | + | − |
| Deletion mutant | 7 kb flanks | + | + | − |
| Deletion mutant | 7 kb flanks | + | + | − |
| Deletion mutant | 7 kb flanks | + | + | − |
| Deletion mutant | 7 kb flanks | + | + | − |
| Deletion mutant | 7 kb flanks | + | + | − |
| Deletion mutant | 7 kb flanks | + | − | − |
| Deletion mutant | 7 kb flanks | + | + | − |

### Shake Flask Tests

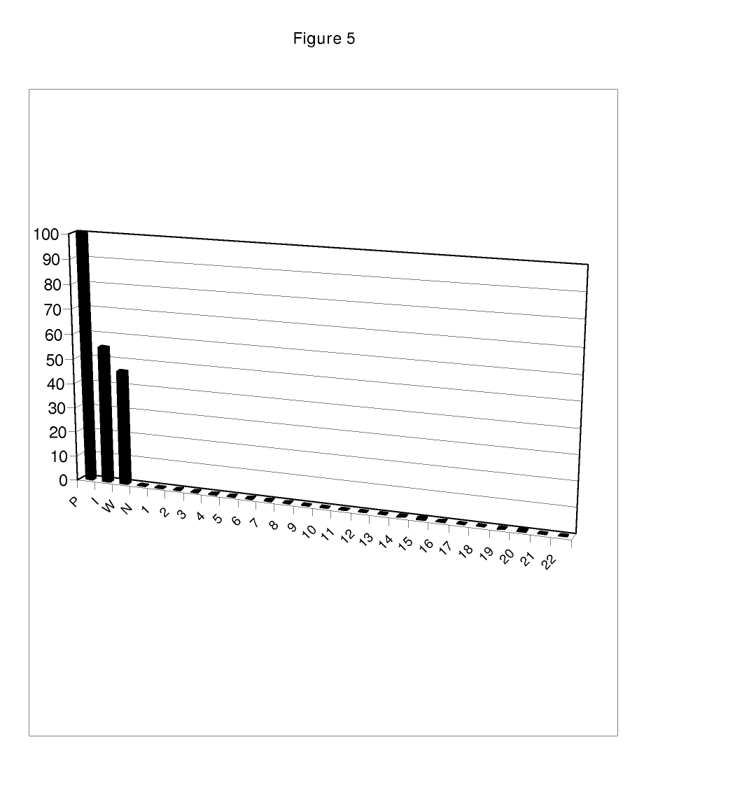

All 22 mutants were tested in shake flask to confirm the penicillin-negative phenotype. For this, the mutants were inoculated in liquid mineral medium with phenyl acetic acid as precursor. Samples were analyzed with NMR. Indeed none of the mutants was capable of producing penicillin G (FIG. 5) and therefore it was concluded that all were correct Penicillium chrysogenum platform strain isolates.

### Example 2

Improved β-Lactam Production in Penicillium chrysogenum Platform Strain

### DNA Fragments

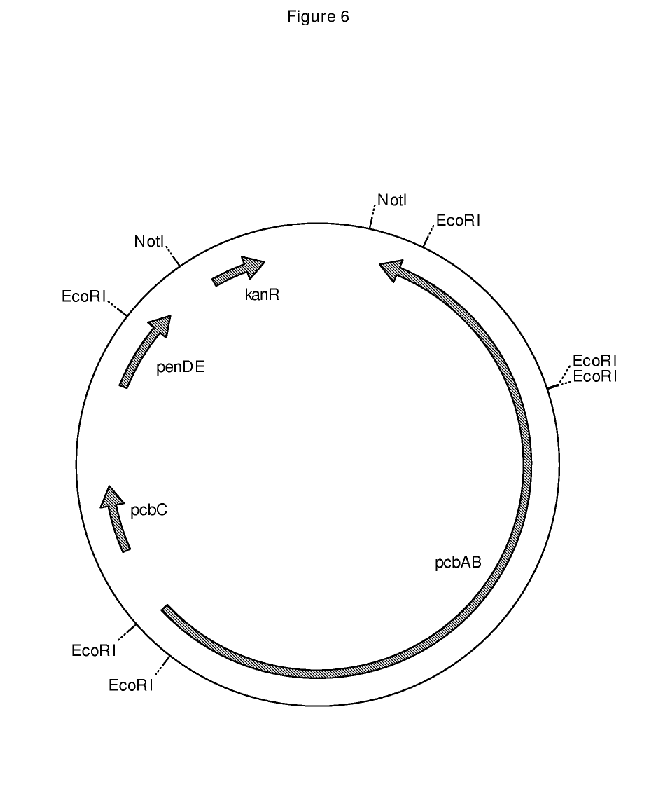

The three biosynthetic genes, pcbAB-pcbC-penDE, were amplified as one fragment using the forward primer (SEQ ID NO 17) and the reverse primer (SEQ ID NO 18), including Gateway recombination sequences (Invitrogen). The 17 kb PCR fragment was cloned in pCRXL (Invitrogen) and using the Gateway system (Invitrogen) transferred in to a so-called entry vector, pDONR221-Pcpencluster (see FIG. 6).

### Penicillium Transformation

The 17 kb fragment was isolated from pDONR221-Pcpencluster via NotI-digestion. It was co-transformed to the Penicillium chrysogenum platform strain with a ble expression cassette encoding for phleomycin resistance. This cassette can be isolated as a 1.4 kb Sail fragment from pAMPF7 (F. Fierro et al., 1996, Curr. Genet. 29, 482-489). Phleomycin resistant transformants were isolated and checked for the re-introduction of the three penicillin biosynthetic genes using the bioassay with the Escherichia coli ESS strain. Twelve colonies, which showed clear inhibition on the growth of the Escherichia coli, were selected for further analyses.

### Shake Flask Tests

TABLE 5
Relative penicillin G production after re-introduction of the
biosynthetic gene cluster in Penicillium chrysogenum
platform strain

| | Penicillin G titer |
|---|---|
| Strain | (relative) |
| Parent strain | 100 |
| Penicillin-free strain | 0 |
| Transformant 1 | 1 |
| Transformant 2 | 83 |
| Transformant 3 | 53 |
| Transformant 4 | 54 |
| Transformant 5 | 55 |
| Transformant 6 | 39 |
| Transformant 7 | 40 |
| Transformant 8 | 8 |
| Transformant 9 | 38 |
| Transformant 10 | 42 |
| Transformant 11 | 40 |
| Transformant 12 | 72 |

The 12 transformants with the penicillin biosynthetic gene cluster re-introduced in the Penicillium chrysogenum platform strain were tested in liquid mineral medium supplemented with phenyl acetic acid for their penicillin G production capabilities. All transformants are capable of producing penicillin G, although with variation in the final titer observed (Table 5). This restoration of the penicillin biosynthetic capability confirms that the Penicillium chrysogenum platform strain only lost their penicillin biosynthetic genes and retained all the other mutations making it such a good industrial production strain.

### Strain Analysis

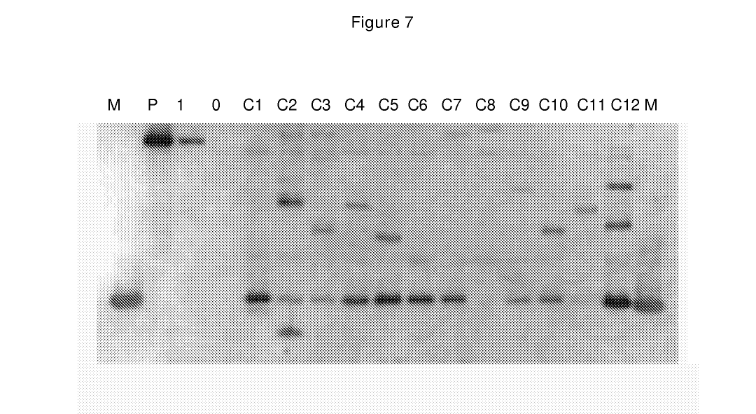

Southern Blot analysis of the obtained Penicillium chrysogenum strains (Table 5, FIG. 7) was carried out. As a probe, an internal DNA fragment of 300 nucleotides from the pcbAB sequence was amplified.

TABLE 6
Primer sequences used to generate probes
for Southern Blot analysis

| Gene | Forward primer | Reverse primer |
|---|---|---|
| pcbAB | SEQ ID NO 19 | SEQ ID NO 20 |

All strains with intact penicillin gene amplicon sequences (i.e. the industrial parent strain as well as the 1 amplicon copy strain) show an 8.1 kb DNA fragment. Penicillium chrysogenum platform strain shows no hybridization with the probe. All randomly penicillin gene-cluster reintegrated strains exhibit a 1.7 kb DNA fragment, which can only be explained by tandem integration of two penicillin gene clusters, head to head, head to tail or tail to tail. These hybridization intensities of the probes, confirmed that 2-4 penicillin gene sets were reintegrated into the genome. In comparison with the penicillin G titers observed, it can be concluded that the penicillin G titers per re-integrated penicillin gene cluster are at least as high as in the parent Penicillium chrysogenum industrial strain. Therefore, the Penicillium chrysogenum platform strain still possesses the conserved beneficial production features for β-lactams and natural products. Moreover, as these fragments integrate at random positions in the genome the results indicate that different genomic loci cause differences in transcription efficiency, ultimately resulting in different penicillin titers. Southern blot analyses show that in some transformants the ratio penicillin G to gene copy number is improved as compared to the industrial parent strain, suggesting that other loci in the Penicillium chrysogenum genome are even more suitable for expression of the penicillin biosynthetic genes as the locus used in the original strains.

### Example 3

High-Titer Statin Production in Penicillium chrysogenum

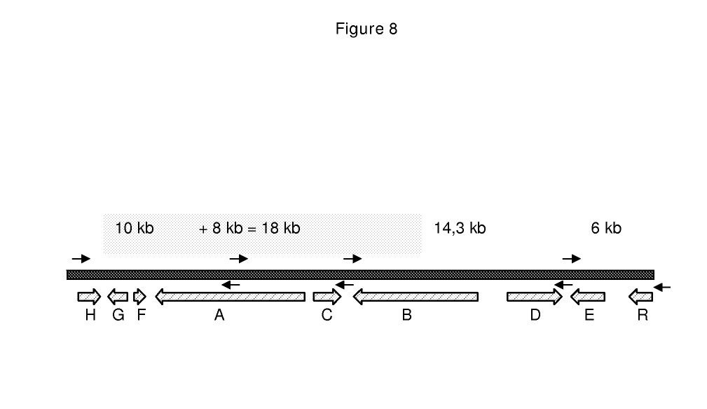

Penicillium citrinum is the natural compactin producer (Y. Abe et al., 2002, Mol Genet Genomics 267, 636-646). The genes encoding the metabolic pathway are clustered in one fragment on the genome (FIG. 8). Several reports in literature describe the functional role of some of these genes in the biosynthesis pathways. Moreover, over expression of the whole cluster or the specific regulator increases the compactin titer (Y. Abe et al., 2002, Mol. Genet. Genomics 268, 130-137; Abe, Y. et al., 2002, Mol. Genet. Genomics, 268, 352-361). However the production titers of both the wild type strains and the recombinant strains are still very low, so a better production host would be favorable.

### Cloning the Compactin Cluster

TABLE 7
Oligonucleotides used to amplify the
compactin biosynthetic gene cluster

| | Forward primer | | Reverse primer | |
|---|---|---|---|---|
| | Cluster | Gateway | Cluster | Gateway |
| Target DNA | sequence | Sequence | sequence | Sequence |
| Left part of the | SEQ ID NO 21 | attB4 | SEQ ID NO 22 | — |
| compactin | | | | |
| cluster (10 kb | | | | |
| fragment) | | | | |
| Left part of the | SEQ ID NO 23 | — | SEQ ID NO 24 | attB1 |
| compactin | | | | |
| cluster (8 kb | | | | |
| fragment) | | | | |
| Internal 6 kb of | SEQ ID NO 25 | — | SEQ ID NO 26 | — |
| left part of | | | | |
| compactin | | | | |
| cluster | | | | |
| Middle part of | SEQ ID NO 27 | attB1 | SEQ ID NO 28 | attB2 |
| the compactin | | | | |
| cluster (14 kb | | | | |
| fragment) | | | | |
| Right part of | SEQ ID NO 29 | attB2 | SEQ ID NO 30 | attB3 |
| the compactin | | | | |
| cluster (6 kb | | | | |
| fragment) | | | | |

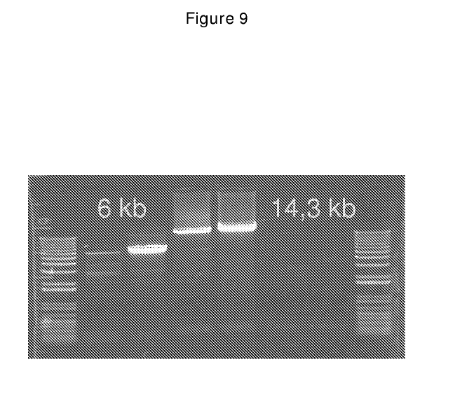

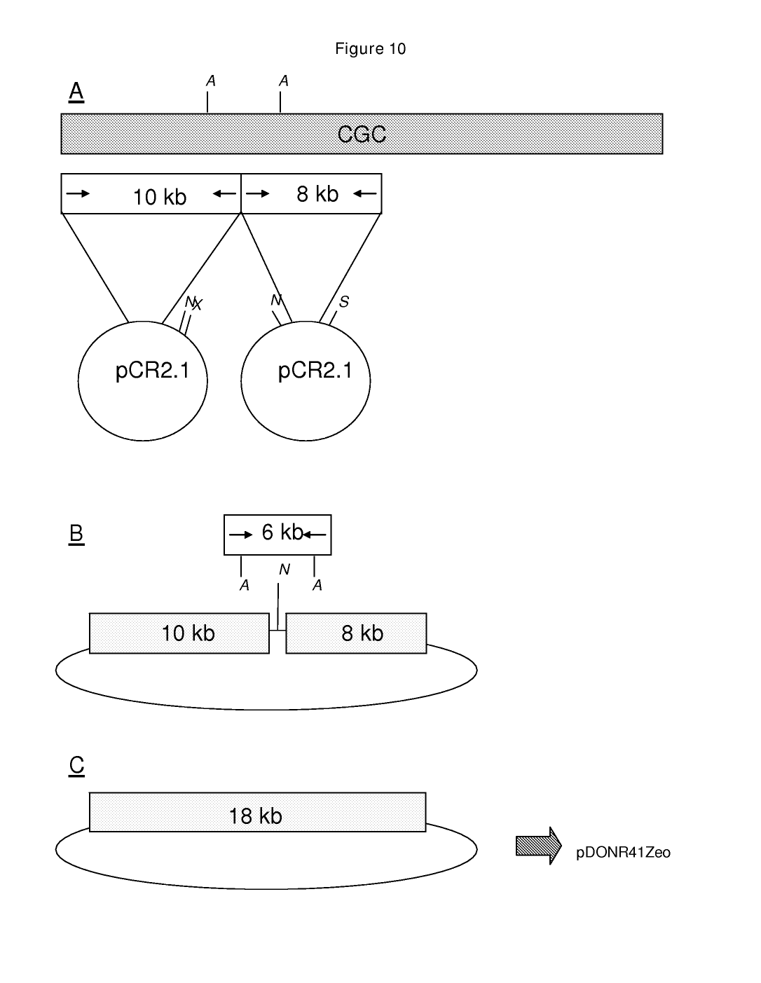

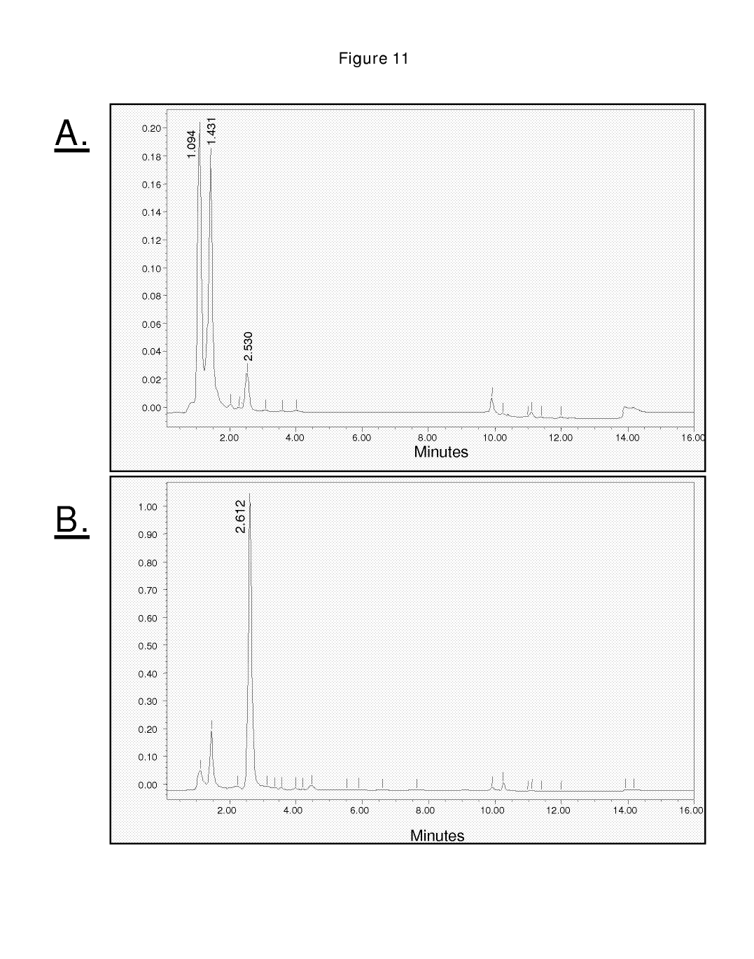

Chromosomal DNA was isolated from Penicillium citrinum NRRL8082. As the full gene cluster is difficult to amplify via PCR due to its size (38 kb), the gene cluster was divided in three fragments: one of 18 kb, one of 14 kb and one of 6 kb. The middle and right part, i.e. the 14 and 6 kb fragments, were readily PCR amplified (FIG. 9) and cloned using Gateway (Invitrogen) into the entry vectors pDONRP4-P1R and pDONR221 with a so-called LR gateway reaction. This was done according to the suppliers' instructions. The 18 kb fragment was cloned in a two-step procedure. First, a 10 and an 8 kb fragment were amplified. Both fragments were cloned separately in pCR2.1 TOPO T/A (Invitrogen) and subsequently fused together via restriction enzyme cloning and ligation (see FIG. 10 for details). Finally, the fragment was transferred to the pDONR41Zeo vector using Gateway technology. The amplified fragments were verified via sequencing. Using a so-called Multi-site Gateway Reaction (see manual Invitrogen) these three gene fragments containing all the genes of the compactin biosynthetic gene clusters can be recombined into one fragment, spanning the whole region.

### Penicillium Transformation

The three compactin gene cluster fragments were co-transformed to the Penicillium chrysogenum platform strain with a ble expression cassette encoding for a protein that mediates phleomycin resistance. This cassette can be isolated as a 1.4 kb SalI fragment from pAMPF7 (F. Fierro et al., 1996, Curr. Genet. 29, 482-489). Selection of transformants was done on mineral medium agar plates with 50 μg/ml phleomycin and 1 M saccharose for osmotic stability. Phleomycin resistant colonies were re-streaked on fresh phleomycin agar plates w/o the saccharose and grown until sporulation. The phleomycin resistant transformants were screened via colony PCR for the presence of one or more compactin gene fragments. For this, a small piece of colony material was suspended in 50 μl TE buffer according to standard DNA procedures and incubated for 10 min at 95° C. To discard the cell debris the mixture was centrifuged for 5 minutes at 3000 rpm. The supernatant (5 μl) was used as a template for the PCR-reaction with SUPER TAQ from HT Biotechnology Ltd. The PCR-reactions were analyzed on the E-gel96 system from Invitrogen. First, the presence of the 18 kb fragment was checked. Out of 480 colonies checked 112 had the 18 kb fragment stably integrated (˜23%). Subsequently, the presence of the other two fragments (14 and 6 kb) was verified. 45 of the 18 kb-positive transformants also had both other parts of the compactin gene cluster and thereby qualified as putative compactin production strains.

TABLE 8
Oligonucleotides used in colony PCR for determining the
presence of the compactin biosynthetic gene cluster

| Target DNA | Forward primer | Reverse primer |
|---|---|---|
| 18 kb fragment | SEQ ID NO 31 | SEQ ID NO 32 |
| 14 kb fragment | SEQ ID NO 33 | SEQ ID NO 34 |
| 6 kb fragment | SEQ ID NO 35 | SEQ ID NO 36 |
| niaA | SEQ ID NO 3 | SEQ ID NO 4 |

### Shake Flask Tests

The Penicillium chrysogenum platform strain transformants with the full compactin gene cluster were evaluated in liquid mineral media (without phenyl acetic acid) for the presence of (hydrolyzed) compactin and ML-236A (6-(2-(1,2,6,7,8,8a-hexahydro-8-hydroxy-2-methyl-1-naphthalenyl)ethyl)tetrahydro-4-hydroxy-2H-pyran-2-one). After 168 h of cultivation at 25° C. in 25 ml the supernatant was analyzed with HPLC using the following equipment and conditions:
Column: Waters XTerra RP18
Column Temp: Room temp.
Flow: 1 ml/min
Injection volume: 10 μl
Tray temp: Room temp.
Instrument: Waters Alliance 2695
Detector: Waters 996 Photo Diode Array
Wavelength: 238 nm
Eluens: A: 33% CH₃CN, 0.025% CF₃CO₂H in milliQ water; B: 80% acetonitrile in milliQ water; C: milliQ water
Two different gradients were used:

Gradient 2

| | |
|---|---|
| Time | Eluens (%) |

| (min) | A | B | C |
|---|---|---|---|
| 0.0-5.0 | 50 | 0 | 50 |
| 5.0-5.1 | 50→100 | 0 | 50→0 |
| 5.1-9.0 | 100 | 0 | 0 |
| 9.0-9.1 | 100→0 | 0→100 | 0 |
| 9.1-13.0 | 0 | 100 | 0 |
| 13.0-13.1 | 0→50 | 100→0 | 0→50 |
| 13.1-15.0 | 50 | 0 | 50 |

Gradient 1

| | |
|---|---|
| Time | Eluens (%) |

| (min) | A | B | C |
|---|---|---|---|
| 0.0-8.0 | 100 | 0 | 0 |
| 8.0-8.1 | 100→0 | 0→100 | 0 |
| 8.1-12 | 0 | 100 | 0 |
| 12.0-13.0 | 0→100 | 100→0 | 0 |
| 13.0-14.0 | 100 | 0 | 0 |

| | | |
|---|---|---|
| Retention times when | Hydrolyzed compactin | 10.4 min |
| using gradient 1: | Compactin | 10.9 min |
| | ML-236A | 2.6 min |
| Retention times when | Hydrolyzed compactin | 11.5 min |
| using gradient 2: | Compactin | 11.8 min |
| | ML-236A | 8.6 min |

The wild type Penicillium citrinum strains barely produce any statins, while the Penicillium chrysogenum transformants produce significant amounts (Table 9). An example of the HPLC chromatograms is shown in FIG. 11.

TABLE 9
Statin Levels (compactin and ML-236A)
produced by different strains.

| | Compactin | |
|---|---|---|
| Strain | (mg/L) | ML-236A (mg/L) |
| Penicillium citrinum NRRL8082 | <0.5 | 0 |
| Penicillium citrinum NRRL8082 | <0.5 | 0 |
| Penicillium citrinum NRRL8082 | 0.9 | 0 |
| Penicillium citrinum NRRL8082 | <0.5 | 0 |
| Penicillium chrysogenum platform | 0 | 0 |
| strain | | |
| Penicillium chrysogenum platform | 0 | 0 |
| strain | | |
| Compactin cluster transformant | 10 | 465 |
| Compactin cluster transformant | 7 | 420 |

This data confirms the high potential of using isolates of Penicillium chrysogenum industrial production strains as platform strains for the production of API's or API-building blocks.

1. Recombinant Penicillium chrysogenum strain characterized in that a gene selected from the list consisting of penDE, pcbAB and pcbC is inactivated.

2. Strain according to claim 1 derived from a strain producing more than 1.5 g/L β-lactam after 96 h fermentation on complex medium.

3. Strain according to claim 1 wherein said gene is pcbC.

4. Strain according to claim 3 further lacking or having an inactivated gene pcbAB and/or penDE.

5. Strain according to claim 1 further comprising a gene involved in the biosynthesis of a compound of interest.

6. Method for producing a compound of interest in a eukaryotic recombinant microorganism comprising the steps of:
(a) Reducing the level of secondary metabolite production in said microorganism with 50-100%;
(b) Introducing a heterologous gene into said microorganism

7. Method according to claim 6 wherein said wherein said compound of interest is not a polypeptide or said secondary metabolite is not trichothecene.

8. Method according to claim 6 wherein said microorganism is a β-lactam producing microorganism.

9. Method according to claim 8 wherein said β-lactam producing microorganism is from the genus Penicillium.

10. Method according to claim 9 wherein step (a) is carried out by performing the steps of:
(a.1) Isolating an isolate with a single genomic copy of the penicillin gene cluster from a Penicillium strain
(a.2) Inactivating gene pcbC from the isolate obtained in step (a.1)
(a.3) Optionally inactivating genes pcbAB and/or penDE from the isolate obtained in steps (a.1) or (a.2)

11. Method according to claim 10 wherein said inactivation in steps (a.2) and/or (a.3) is performed by deletion.

12. Method according to wherein said compound of interest is a secondary metabolite.

13. Use of a strain according to claim 1 for the preparation of a compound of interest.

14. Use according to claim 13 wherein the compound of interest is produced via enzymes encoded by recombinant genes.

15. Use according to claim 13 wherein the compound of interest is aflatoxin, aphidicolin, compactin, ergotamine, fumonisin, lovastatin, lysergic acid, paxicillin, trichothecene or 6-(2-(1,2,6,7,8,8a-hexahydro-8-hydroxy-2-methyl-1-naphthalenyl)ethyl)tetrahydro-4-hydroxy-2H-pyran-2-one.

16. Use according to claim 13 wherein the compound of interest is suitable for conversion into compactin and/or pravastatin.

17. Use of a strain according to claim 1 for the assessment of the biological function of genes.
